(19) United States
(12) Patent Application Publication
(10) Pub. No.: US 2017/0372251 A1
(43) Pub. Date: Dec. 28, 2017

(54) SYSTEM FOR SIMULATING SENSORS

(71) Applicant: SICK AG, Waldkirch (DE)

(72) Inventors: Gerhard ALT, Waldkirch (DE); Patrick BORNSTEIN, Waldkirch (DE); Davorin JAKSIC, Waldkirch (DE); Daniel KIETZ, Waldkirch (DE); Christoph MÄRKLE, Waldkirch (DE); Michael BURGER, Waldkirch (DE); Armin VOGELBACHER, Waldkirch (DE); Michael STINGL, Waldkirch (DE); Simone SCHWARZ, Waldkirch (DE)

(21) Appl. No.: 15/631,209

(22) Filed: Jun. 23, 2017

(30) Foreign Application Priority Data
Jun. 24, 2016 (EP) 16176234.9

(51) Int. Cl. G06Q 10/06

(52) U.S. Cl. CPC G06Q 10/06393; A61B 2562/06

(57) ABSTRACT

The invention relates to a system for simulating sensors that in particular measure a distance between the sensor and an object, that measure geometrical dimensions of the object, that measure positions of the object, that measure material, contrast, color, luminescence, brightness or transparency of the object, that measure polarization of the light reflected by the object or that measure a magnetic field strength.

The invention relates to a system for simulating sensors that in particular measure a distance between the sensor and an object, that measure geometrical dimensions of the object, that measure positions of the object, that measure material, contrast, color, luminescence, brightness or transparency of the object, that measure polarization of the light reflected by the object or that measure a magnetic field strength.

Sensor manufacturers offer a plurality of sensors such as light barriers, reflected light barriers, multibeam light barriers, light scanners, light grids, optical line sensors, camera systems, inductive or capacitive proximity switches, magnetic cylinder sensors, magnetic proximity sensors, radar distance sensors, and possibly also further other measuring devices. Every single type is in this respect also available in multiple variants. For example, a simple light barrier can be offered in variants that bridge different distances, that work at different wavelengths, that work at different powers, etc., etc. Sensor manufacturers therefore have ranges of many hundreds or many thousands of different sensors and measuring units. All these sensors and measuring units will simply be called “sensors” in the following.

There is therefore the problem for a customer who wants to set up a new machine, for example, and who requires specific individual ones of this multitude of sensors for his purpose, where the customer himself does not know all the details of the sensors, their performance capabilities and the range of possible applications, of finding the right, suitable sensors for his application.

Since large databases can today be accessed using current computers and even in a mobile manner via the internet, so-called product finders are known with which stored sensor properties can be selected step-by-step from a drop-down menu in a program, for example, so that single sensors or a plurality of sensors are listed at the end of the possible choices.

However, this is merely a selection using the data sheets of the sensors. Whether the sensors selected in this manner also satisfy their function in the planned application in turn depends on the technical knowledge the person making the selection has with respect to the application and with respect to the sensors and on how intelligently this person can link up this technical knowledge.

Starting from this prior art, it is the object of the invention to provide a system with which the sensors themselves, that is their function in an application, can be simulated with the aim of testing which sensor satisfies or best satisfies the application.

This object is satisfied by a system in accordance with the invention for simulating sensors. The system comprises:
an input unit for inputting a limited number of physical parameters that parameterize one or more predefined sensor requirements;
a memory unit in which sensor models can be stored with sensor model properties that correspond to the sensors;
an evaluation unit for simulating all the sensors as to whether they satisfy the parameterized sensor requirements and the simulation takes place with reference to the stored sensor models, with at least one mapping specification being associated with each sensor model property, with which mapping specification one or more of the sensor model properties are mapped onto one of the parameterized sensor requirements;
and an assessment unit that assesses whether a sensor model satisfies the parameterized sensor requirements.

This object is also satisfied by a corresponding computer program and computer program product.

The sensor is therefore simulated by a suitable simulation model in a described application environment. The particular advantage of the invention is that an association of sensor models to the predefined requirements of an application takes place by the simulation that is optimum for the application, with the sensor models comprising all those which are present in the memory unit. Not only all the sensors are thus considered, so that always the most suitable sensors or sensor can be found by the simulation, but also no extensive special knowledge of the sensors is required for the application of the simulation. Due to the stored sensor models, the user who wants to use the sensors in his application does not require any special technical knowledge of the sensors. The “best match” between the application and the sensor is automatically found by the simulation. The mapping specifications specific to the sensor model primarily serve this purpose. The expert knowledge of the sensor models and of the application fields is contained in these mapping specifications. The big advantage is that this expert knowledge only has to be used once at the time of the setting up of the mapping specifications and is subsequently always available again without any effort for every simulation. A substantial effort and/or cost is/are thus saved. The expert knowledge that is contained in the named mapping specifications is also so-to-say conserved with the system in accordance with the invention and can be added to by further sensor model properties over time by adding further sensor models. It is not required for the person who carries out the simulation to have this expert knowledge by himself, he only utilizes it without having to have the knowledge himself.

Projects such as the equipping of large machines with a plurality of different sensors can be processed considerably faster using the system in accordance with the invention and indeed by persons who do not themselves have expert knowledge. This time gain is the larger, the larger the total application is, e.g. whole production lines.

In a further development of the invention, it cannot only be determined by the simulation whether a sensor satisfies the parameterized sensor requirements, but an assessment can also be made by the assessment unit as to how well a sensor model satisfies the parameterized sensor requirements. A ranking of the simulated sensors can thus be output corresponding to the assessment result so that the user gets to know those sensors best matching the application.

A display unit is provided in a further development of the invention to output the simulation result to display at least some of the assessment results. The ranking can e.g. thus be displayed.

In a further development of the invention, an analytical function and/or a diagram stored in the database and/or a look-up table can be stored as the mapping specification. The true expert knowledge is contained therein. An example should illustrate this. For example, the requirement may be made in the application that a barcode of a minimal line thickness d and with a line spacing d1 should be detected at a contrast K and at a predefined speed v within a distance range. A stored mapping specification for a point scanning sensor could comprise the linking of different functional relationships such as the light spot size over the scanning distance, the light spot size in relation to the line width and to the gap and the ratio of the contrast resolution of the sensor to the prevailing contrast of the application (barcode). The advantage of the invention is then exactly that such complex functional relationships can be taken into account by the stored mapping specifications.

The mapping specifications can also comprise the simplest case when namely a single sensor model property is identical to a sensor requirement. This is admittedly not the subject of the invention, but should nevertheless be mentioned here to show that the invention can also cover what is known. In this case, the mapping specification is a 1 to 1 association. This can e.g. be the case when the sensor model property is a distance range within which the sensor can work and the associated sensor requirement is the distance to be measured between the sensor and the object. The same physical parameter, namely the distance, then forms the background to the sensor model property and the parameterized sensor requirement.

In a further development of the invention, the simulation variety that results from the number of stored sensor models and from the associated sensor model properties can be reduced in advance. That is in that a pre-filter is provided that can be selected before the start of the simulation and via which a pre-selection of the sensor models takes place. An example would e.g. be the selection of the physical principle, that is whether the sensor works magnetically, optically or capacitively. It is thus conceivable that a user of the simulation system has prior knowledge and already knows beforehand which general kind of sensor model can be considered, such as an optical sensor, an inductive sensor or a capacitive sensor. Such a pre-filtering considerably reduces the simulation variety and therefore produces a result faster.

Other pre-filters that also have the purpose of a reduction of the simulation variety are conceivable. A selection can thus be polled with the pre-filter, for example, with respect to the temperature, contamination and moisture that then likewise allow a localization.

In the same way, a post-filter can be selectable after the simulation, that is after receiving the simulation result, by which further parameters can be fixed such as the type of plug, a pnp or npn output, line length, etc. that do not require a simulation.

The invention thus also relates to a simulation method for simulating sensors that in particular measure a distance between the sensor and an object, that measure geometrical dimensions of the object, that measure positions of the object, that measure material, contrast, color, luminescence, brightness or transparency of the object, that measure polarization of the light reflected by the object or that measure a magnetic field strength. The simulation method that is carried out on the above-described system comprises the steps:
inputting a limited number of physical parameters that parameterize one or more predefined sensor requirements into an input unit of the system;
simulating the sensors by means of sensor models that are stored in a memory unit and have sensor model properties,
with the simulation taking place in an evaluation unit with reference to the stored sensor models, with at least one mapping specification being associated with each sensor model property and with which mapping specification one or more of the sensor model properties are mapped onto one of the parameterized sensor requirements; and
assessing in an evaluation unit of the system whether a sensor model satisfies the parameterized sensor requirements.

In a further step, an assessment can additionally be carried out as to how well a sensor model satisfies the parameterized sensor requirements. A results ranking can thus be output as to which sensor model and thus which sensor best satisfies the requirements.

The invention will be explained in detail in the following with reference to an embodiment and to the drawing. There are shown in the drawing:

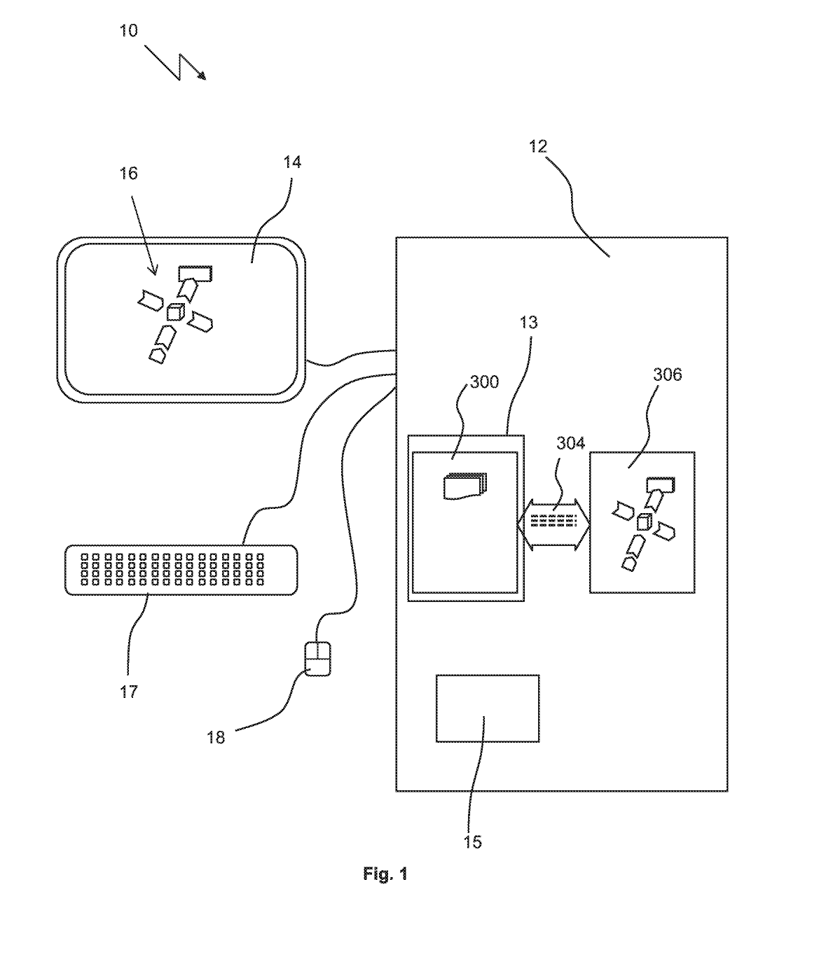

FIG. 1 a schematic representation of a system in accordance with the invention;

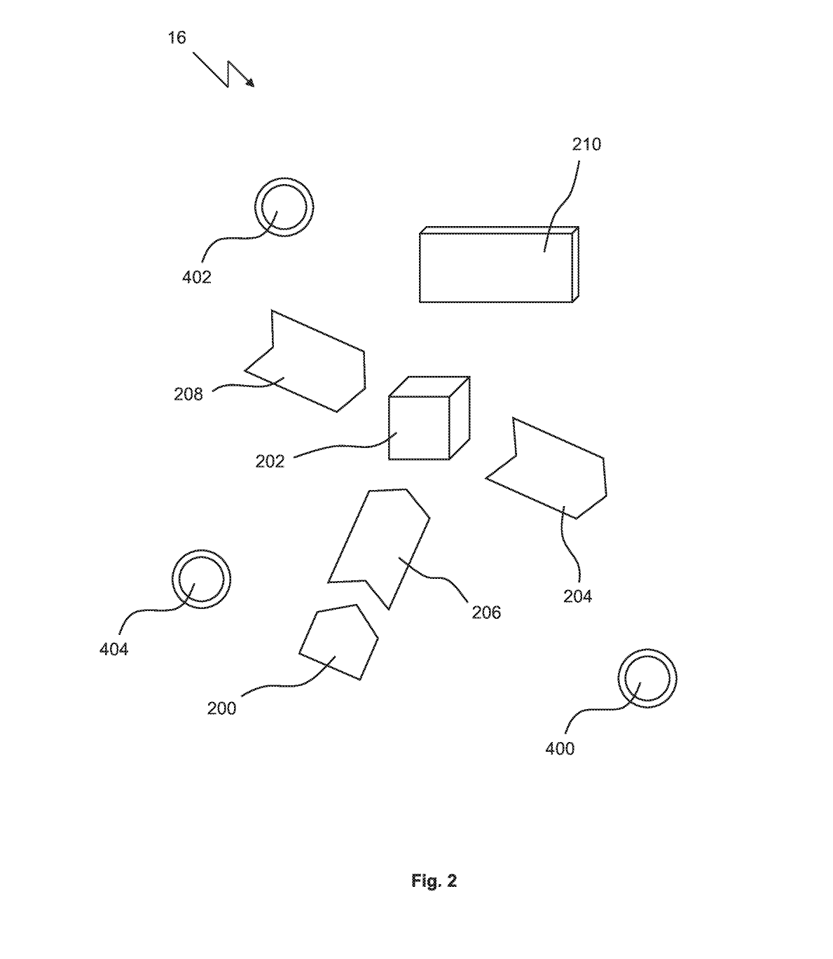

FIG. 2 a schematic representation of a stylized graphic for inputting parameters of predefined sensor requirements; and

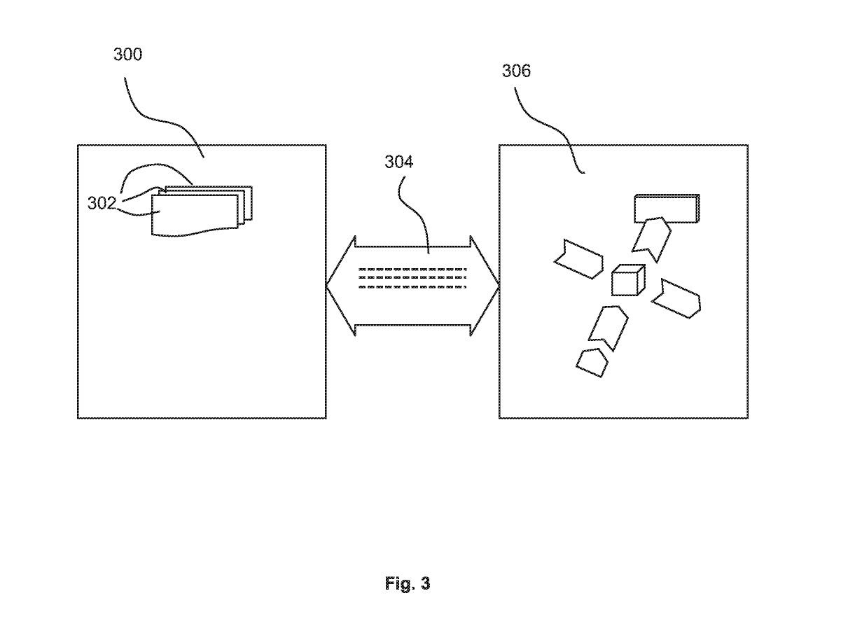

FIG. 3 a schematic representation for illustrating the mapping specifications for associating the sensor model properties with the parameterized sensor requirements.

Sensors suitable for a current application should ultimately be determined from a plurality of sensors using the system in accordance with the invention for simulating sensors. Sensor models are therefore simulated by the simulation while taking account of application-specific sensor requirements that result from a current application. The application-specific sensor requirements are determined by physical parameters. In the simplest case, an application-specific sensor demand can, for example, be that the sensor has to be able to measure in a distance range of 1 m to 2 m. The parameter that parameterizes this sensor requirement is then a distance value.

The system 10 can be formed by a computer 10 that can be configured in the most varied, known manners, for example as a desktop PC as shown by way of example in FIG. 1, as a tablet PC, as a smartphone or the like. The system 10 has at least one input unit 17, 18, a memory unit 13 and an evaluation unit 12 as well as a display unit 14. Other compositions of the system 10 are possible. The memory unit could e.g. be installed on an external server (“cloud”).

For support in the inputting of the parameters, a stylized, typical exemplary application situation 16 is shown on the display unit 14 by means of graphical symbols that will be explained in the following. This exemplary application situation 16 is representative for a plurality of applications so that it is of a stylized nature. The individual symbols represent physical objects and/or properties of the application. The aim of the representation is ideal graphical support in the detection of the parameters for a sensor requirement.

The plurality of parameters may not ask too much of the user and require an ideally adapted graphical support in order e.g. to detect object dimensions correctly with respect to the position of the sensor and to the direction of movement of the object. The application scene is shown suitably when it reproduces the actual situation as correctly as possible and causes a high identification in the user.

The exemplary application situation 16 is shown enlarged in FIG. 2 and will be explained in more detail with respect to FIG. 2. A symbol 200 is representative of the sensor (or sensors) that should operate the current application. The application itself is represented by symbols 202, 204 and 206. The symbol 202 is representative of an object to be detected; the symbol 204 is representative of a direction of movement of the object and the symbol 206 is representative of a distance between the sensor 200 and the object 202. The distance itself, but also foreground information such as the installation conditions can also be input via the symbol 206 as explained further below.

Further symbols can additionally be provided such as a symbol 208 for a performance requirement, which is to be understood as the object speed, the resolution and the accuracy, and a symbol 210 for the description of the space behind the object (application background).

Parameter sets are defined for each symbol of the application, with the parameters corresponding, as mentioned above, to sensor requirements, that is to physical properties of the current application for which a sensor is to be simulated and thus specifying the application. The parameter sets can be preallocated with suitable default values.

An object parameter set is defined with respect to the object symbol 202 that can comprise one or more of the following parameters: “minimal object length in the direction of movement”, “maximum object length in the direction of movement”, “minimal object width”, “maximum object width”, “minimal object height”, “maximum object height”, “positional tolerance”, “material”, “contrast”, “color”, “luminescence”, “brightness”, “transparency”, “depolarization capability”, “focusing capability”, “magnetic field strength”.

To be able to correctly detect the object dimensions with respect to the sensor and to the direction of movement, graphical representations are sensible that are arranged downstream and with which the length, width and height of objects is also clearly enabled with two-dimensional sensor devices. This is roughly indicated by the three-dimensional representation of the object system 202 as a box.

A direction parameter set is defined with respect to the direction of movement symbol 204 and a distance parameter set is defined with respect to the object distance symbol 206. The distance parameter set can comprise one or more of the following parameters: “distance sensor to object”, “distance sensor to reflector”, and “object guidance tolerances”. The properties of the space in front of the object (foreground information) and/or installation conditions can also be input via the object distance symbol 206. The installation conditions are in particular relevant to inductive sensors since what is decisive with these sensors is the distance at which objects, in particular metal objects are located, and the size of said objects, in the region of the front end of the sensor.

The aforesaid background parameter set can comprise a parameter “background type”. Different background types of different sensor systems can thus be described. The background is thus formed by the receiver in a separate transmitter/receiver system. With fork sensors, the background is formed by one of the fork branches; and with reflection systems by a reflector. Another parameter can be “extraneous light”. Background properties such as disturbing extraneous light or undefined reflections at boundary layers can thereby also be described from which, for example, the selection of specific light scanners (with or without background suppression) is derived.

The larger part of all possible applications can be detected using this set of symbols and associated parameters. The parameters are stored in a parameter memory 306. The input can take place through the most varied known types, for example by clicking on a symbol, whereby a drop down selection menu opens, or by the input of specific numbers, e.g. input of the sensor to object distance, for example by direct number input into a corresponding window that opens by clicking on the corresponding symbol 206 or by dragging graphical symbols.

Sensor models are stored in the memory unit 13 with sensor model properties. The invention relates to the situation when the parameterized sensor requirements are not identical to sensor model properties since a mechanism is required for exactly this situation to bring the sensor requirements and the sensor model properties into line. This does not preclude there nevertheless being able to be parameterized sensor requirements that correspond 1 to 1 to a sensor model property. This applies, for example, to a sensor to object distance as a sensor requirement that corresponds to a sensor model property “measurement range”. This is, however, a trivial case for which no simulation would be required on its own.

The invention therefore deals with the situations in which these trivial cases are not present or are not present alone. The evaluation unit 12 is therefore provided with which all the stored sensors can be simulated. A check is therefore made in the simulation whether the available sensors satisfy the parameterized sensor requirements when applying the associated sensor model. Since, as already mentioned, the parameterized sensor requirements are not identical to the sensor model properties, the simulation takes place in accordance with the invention in a manner such that at least one mapping specification is associated with each sensor model property, with which mapping specification one or more of the sensor model properties is mapped to one of the parameterized sensor requirements.

This is shown in a simple manner in FIG. 3. In this respect, the sensor model properties 302 that are stored in a database 300 are mapped to the parameters of the application stored in the parameter memory 306 by means of the mapping specification 304 associated with the respective sensor model. The mapping specifications 304 specific to the sensor model had been previously set up. This means that there are associations between the stored parameters and the sensor model properties 302 on the basis of the stored mapping specifications 304. After the selection or input of the parameters by an input at the computer 10, the sensor modules are now run through the simulation by the computer 10.

The mapping specifications 304 were set up with knowledge of all the selectable parameters and thus include the expert knowledge of the sensors and application fields. A mapping specification 302 can be stored as an analytical function and/or as a diagram stored in the database and/or as a look-up table. For example, the requirement may be made in the application that a barcode of a minimal line thickness d and with a line spacing d1 should be detected at a contrast K and at a predefined speed v within a distance range. A stored mapping specification for a point scanning sensor could comprise the linking of different functional relationships such as the light spot size over the scanning distance, the light spot size in relation to the line width and to the gap and the ratio of the contrast resolution of the sensor to the prevailing contrast of the application (barcode). The advantage of such mapping specifications is actually that complex functional relationships can be taken into account in the simulation.

The expert knowledge of the sensors and their usability is included in these more complex mapping specifications that each have to be set up per se once beforehand.

All the sensor modules are therefore run through by the simulation. In accordance with the invention, an assessment unit 15 is finally provided with which an assessment is made as to whether a sensor model satisfies the parameterized sensor requirements. In a further development, the assessment unit 15 can also assess “how well” a sensor model satisfies the sensor requirements. The result is then a “ranking” of sensor models that can be shown on the display unit 14. Sensor models are then displayed that best match the parameterized sensor requirements and thus the application.

In addition to the sensors found, required accessories can also be displayed so that the user can recognize that the accessories displayed are required in addition to the found sensor to satisfy the application. The operation and performance of sensors are namely often directly linked to the specific properties of associated components “accessories”. The sensors and the “accessories” then form a total system. Systems are therefore found in such cases on the basis of the parameters that comprise the actual sensor and further components. For example, the system properties are influenced by “accessories” such as reflectors (size and technology) as well as light guides of different lengths (damping) or specific magnets (magnetic field strength). In addition to the sensors found, associated accessories should therefore also be displayed in the results display. For example, a specific reflection light barrier could be present as the result that has a different range, namely a much larger range, with a triple reflector as the reflector than with a reflection film as the reflector. The additional indication of the required reflector type is therefore sensible and helpful in such a case.

Furthermore, a so-called pre-filter can be provided that can be activated via a symbol 400 or 402. The symbols 400 and 402 stand for two different types of pre-filter.

The one pre-filter 400 serves to restrict the possibilities by a few specific feature queries (parameters) and to simplify the application detection by more specific queries from the a prior knowledge of the specific technology. If the user restricts himself to such specific parameters and if these parameters are in a predefined association with the sensor models, the system can only utilize these associated sensor models for the simulation and can discard other sensor models. The extent of the simulation is thereby reduced and the simulation runs considerably faster. It is therefore meaningful if the symbols of the exemplary application situation 16 can only be activated when such a pre-filter has been run through.

Furthermore, another pre-filter 402 can be provided that corresponds to an expert function or advanced function. This pre-filter requires knowledge of the sensor models since it allows a reduction of the selection of possible sensor models. The expert user can thus directly select sensor models that the simulation should utilize. This in turn reduces the simulation effort and the processing time and, due to the aforesaid associations of the sensor demands with the sensor models, it can also reduce the set of the parameters to be input. This pre-filter is a kind of short cut to the desired technology for the advanced user. It is therefore possible to select one or more of the sensor models such as optical sensors or inductive sensors directly with this short cut. All the other sensor models are then no longer considered in the simulation.

After the simulation, a so-called post-filter can be selectable via a symbol 404. Further parameters such as the type of plug, pnp or npn output, line length and the like can be fixed with this post-filter. A further post-filter can also be provided with respect to features that no longer relate to the current application, but rather relate to non-sensor specific customer wishes such as product novelty, price, availability, special regional features and the like.

1. A system for simulating sensors, the system
having an input unit for inputting a limited number of physical parameters that parameterize one or more predefined sensor requirements;
having a memory unit in which sensor models are stored with sensor model properties;
and having an evaluation unit for simulating all the sensors as to whether they satisfy the parameterized sensor requirements and the simulation takes place with reference to the stored sensor models, with at least one mapping specification being associated with each sensor model property, with which mapping specification one or more of the sensor model properties are mapped onto one of the parameterized sensor requirements,
and the evaluation unit comprises an assessment unit that assesses whether a sensor model satisfies the parameterized sensor requirements.

2. The system in accordance with claim 1, wherein the simulated sensors are configured to measure one of the following: a distance between the sensor and an object, geometrical dimensions of an object, positions of an object, material of an object, contrast of an object, color of an object, luminescence of an object, brightness of an object, transparency of an object, polarization of light reflected by an object and a magnetic field strength.

3. The system in accordance with claim 1, wherein the assessment unit assesses how well a sensor model satisfies the parameterized sensor requirements.

4. The system in accordance with claim 1, wherein a display unit is provided for displaying at least some of the assessment result.

5. The system in accordance with claim 3, wherein the display unit outputs a ranking of the simulated sensors in accordance with the assessment result.

6. The system in accordance with claim 1, wherein the mapping specification is an analytical function and/or a diagram stored in the memory unit and/or a look-up table.

7. The system in accordance with claim 1, wherein only predefined values can be input with respect to specific parameters.

8. A computer program having program code means that are configured such that a system
having an input unit for inputting a limited number of physical parameters that parameterize one or more predefined sensor requirements;
having a memory unit in which sensor models are stored with sensor model properties;
and having an evaluation unit for simulating all the sensors as to whether they satisfy the parameterized sensor requirements and the simulation takes place with reference to the stored sensor models, with at least one mapping specification being associated with each sensor model property, with which mapping specification one or more of the sensor model properties are mapped onto one of the parameterized sensor requirements,
and the evaluation unit comprises an assessment unit that assesses whether a sensor model satisfies the parameterized sensor requirements, arises when the program is executed on a computer.

9. A computer program product having program code means that are stored on a computer-readable data carrier and that are configured such that a system
having an input unit for inputting a limited number of physical parameters that parameterize one or more predefined sensor requirements;
having a memory unit in which sensor models are stored with sensor model properties;
and having an evaluation unit for simulating all the sensors as to whether they satisfy the parameterized sensor requirements and the simulation takes place with reference to the stored sensor models, with at least one mapping specification being associated with each sensor model property, with which mapping specification one or more of the sensor model properties are mapped onto one of the parameterized sensor requirements,
and the evaluation unit comprises an assessment unit that assesses whether a sensor model satisfies the parameterized sensor requirements, arises when the program product is executed on a computer.
